(19) United States
(12) Patent Application Publication
(10) Pub. No.: US 2014/0074024 A1
(43) Pub. Date: Mar. 13, 2014

(54) DRUG DELIVERY DEVICE COMPRISING SAFETY LOCK MEMBER

(71) Applicant: SANOFI-AVENTIS DEUTSCHLAND GMBH, Frankfurt am Main (DE)

(72) Inventors: Axel Teucher, Frankfurt am Main (DE); Michael Jugl, Frankfurt am Main (DE)

(73) Assignee: SANOFI-AVENTIS DEUTSCHLAND GMBH, Frankfurt am Main (DE)

(21) Appl. No.: 14/114,769

(22) PCT Filed: May 11, 2012
(86) PCT No.: PCT/EP2012/058709

§ 371 Date: Oct. 30, 2013

(30) Foreign Application Priority Data
May 13, 2011 (EP) 11165947.0

(51) Int. Cl. A61M 5/50

(52) U.S. Cl. CPC A61M 5/5086
USPC 604/111

(57) ABSTRACT

The present invention relates to a drug delivery device for administering a dose of a medicament, comprising of a first housing component; a second housing component releasably interconnectable with the first housing component in a locked configuration, in which the second housing component covers a dispensing end of the device; and a safety lock member to mechanically engage with the first and with the second housing component in order to inhibit unintentional release of first and second housing component.

### CROSS REFERENCE TO RELATED APPLICATIONS

The present application is a U.S. National Phase Application pursuant to 35 U.S.C. §371 of International Application No. PCT/EP2012/058709 filed May 11, 2012, which claims priority to European Patent Application No. 11165947.0 filed May 13, 2011. The entire disclosure contents of these applications are herewith incorporated by reference into the present application.

### FIELD OF DISCLOSURE

The present invention relates to the field of drug delivery devices and in particular to injection devices like pen-type injectors for administering a pre-defined dose of a medicament. In particular, the invention refers to a safety lock member adapted to inhibit unauthorized or unintentional use of the device, e.g. by children.

### BACKGROUND

Drug delivery devices allowing for multiple dosing of a required dosage of a liquid medicament, such as liquid drugs, and further providing administering of the liquid to a patient, are as such well-known in the art. Generally, such devices have substantially the same purpose as that of an ordinary syringe.

Drug delivery devices of this kind have to meet a number of user specific requirements. For instance in case of those with diabetes, many users will be physically infirm and may also have impaired vision. Therefore, these devices need to be robust in construction, yet easy to use, both in terms of the manipulation of the parts and understanding by a user of its operation. Further, the dose setting must be easy and unambiguous and where the device is to be disposable rather than reusable, the device should be inexpensive to manufacture and easy to dispose. In order to meet these requirements, the number of parts and steps required to assemble the device and an overall number of material types the device is made from have to be kept to a minimum.

Typically, the medicinal product or medicament to be administered is provided in a cartridge having a moveable piston or bung mechanically interacting with a piston rod of a drive mechanism of the drug delivery device. By applying thrust to the piston in distal direction, a pre-defined amount of the medicinal fluid can be expelled from the cartridge.

Typically, drug delivery devices and in particular pen-type injectors comprise a multi-component housing. A distal end section which is adapted to be releasably coupled with a needle assembly is typically protected by a protective cap.

Prior to a use of the device, the protective cap has to be released and removed from the body of the device in order to get access to the distal drug dispensing end of the device. After use of the device, a needle assembly releasably connected to a dispensing and distal end section of the drug delivery is to be discarded and the protective cap is to be remounted on e.g. a cartridge holder section. There, the cap is to be interlocked with a proximal housing component of the drug delivery device in order to establish a locked configuration of the housing components.

However, since the drug delivery device contains a medicament it should be non-accessible for unauthorized persons, especially for children. But since pen-type injectors are commonly used in a home medication environment on a regular basis, even several times every day, it is rather difficult to keep such devices out of the reach of children.

It is therefore an object of the present invention to increase patient safety and to inhibit unintentional or unauthorized use of such drug delivery devices. In particular, the invention aims to protect children from improper use of drug delivery devices. The invention therefore serves to protect children for not hurting or stitching themselves with such devices. In this context it is a further aim of the invention to provide a cost-efficient child lock feature that can even be retrofitted to existing drug delivery devices.

### SUMMARY

The drug delivery device according to the present invention is designed for administering a dose of a medicament, preferably but not exclusively by way of injection. The device comprises a first housing component and a second housing component, wherein the two housing components are releasably interconnectable with each other in a locked configuration. In said locked configuration, the second housing component substantially covers a dispensing or distal end of the device. Typically, the second housing component is designed as a protective cap which is to be disconnected and/or dismantled from the drug delivery device prior to setting and/or dispensing of a dose of the medicament.

In typical embodiments, the second housing component covers a distal portion of a pen-type injector, which is adapted to receive and to hold a cartridge filled with the medicament.

Especially in configurations wherein the cartridge holder is connected with a proximal first housing component of the drug delivery device, the second housing component may mechanically interact and engage with the cartridge holder in order to get in a locked configuration with the second housing component.

According to the present invention there is further provided a safety lock member adapted to mechanically engage with the first and with the second housing component in order to inhibit unintentional release of said first and second housing components with respect to each other. The safety lock member is preferably attached to both, first and second housing components, preferably at an outside facing surface portion of first and second housing components. Moreover, the safety lock member traverses an interface section located between and formed by the first and second housing components. Since the safety lock member mechanically and/or operably engages with first and second housing components, dismantling of the second housing component initially requires to transfer the safety lock member into a release configuration, in which the safety lock member gives way to dismantle and to remove the second housing component relative to the first housing component.

Preferably, the safety lock member provides a child lock feature in that the safety lock member itself is not transferable into a respective release configuration by a child. Forces to be exerted to the safety lock member in order to transfer the same into a release configuration typically exceed a maximum force level that can be applied by children.

Hence, the safety lock member itself can be alternately transferred between a lock configuration, in which the safety lock member keeps the second housing component attached to the drug delivery device and its first housing component and a release configuration, in which the second housing component can be detached or dismantled from the drug delivery device.

According to a preferred embodiment, the safety lock member at least partially encloses the outer circumference of the first and the second housing components when in locking configuration. In said locking configuration, the safety lock member traverses the interface of first and second housing components in e.g. longitudinal or axial direction of the drug delivery device.

According to another preferred aspect, the safety lock member itself comprises an upper and a lower clamping member adapted to receive first and second housing components of the drug delivery device, respectively. Typically, both upper and lower members mechanically engage with first and second housing components when in locking configuration. However, in release configuration, only one or even none of upper or lower clamping member may stay in contact with first and second housing components.

According to a further aspect, upper and lower clamping members of the safety lock member are pivot-mounted with respect to each other, wherein the pivot axis extends in longitudinal or axial direction of first and/or second housing components. This way, the clamping member can be transferred between locking and release configuration simply by pivoting upper and lower clamping member with respect to each other.

It is of further benefit, when upper and lower clamping members are releasably interconnectable in the locking configuration. Upper and lower clamping member typically comprise a geometry that matches with the outer geometry of first and second housing components of the drug delivery device. When in interlocked or closed configuration, upper and lower clamping members may be tight fasten around the circumference of first and second housing components. Typically, the clamp formed by first and second clamping members clasps around the interface of first and second housing members when in interlock configuration.

According to a further preferred embodiment, the upper and/or the lower clamping members are positively engaged with the first housing component and with the second housing component when in interlock configuration. The positive engagement of the safety lock member and first and second housing component serves to inhibit relative axial displacement of first and second housing components as well as first and/or second housing components with respect to the safety lock member and its upper and/or lower clamping member.

The positive engagement of the safety lock member with first and second housing components of the drug delivery device can be attained by providing the upper and/or lower clamping member with at least two inwardly protruding protrusions and/or by outwardly extending recesses. Depending on whether the clamping members comprise, e.g. radially inwardly protruding protrusions or radially outwardly extending recesses, the first and/or second housing components of the drug delivery device comprise corresponding recesses and/or protrusions in order to attain a positive engagement of the safety lock member and its clamping members with first and second housing components.

In an alternative embodiment, the upper and/or the lower clamping member may be also frictionally engaged with the first housing component and with the second housing component when in interlock configuration. The frictional engagement of first and/or second housing components with the safety lock member can be provided instead or in combination with a positive engagement provided by mutually corresponding protrusions and recesses.

In this context it is also conceivable, that a mechanical engagement of second housing components and the safety lock member is attained by way of a positive engagement whereas a mechanical engagement of safety lock member and first housing component is of frictional type.

In particular with a frictional engagement of safety lock member and first and/or second housing components it is of benefit, that the housing components do not have to be modified in order to engage with the safety lock member. Frictional engagement means can therefore be exclusively implemented with the safety lock member, especially with its upper and/or lower clamping member. This way, also existing drug delivery devices can be easily retrofitted with the safety lock member if required.

With embodiments featuring a frictional interlock of safety lock member and housing components it is of further benefit, when the upper and/or lower clamping members at an inside facing surface comprise at least one surface portion having an elastomeric or rubber type material which is adapted to get in frictional contact with an outer surface of first and/or second housing component. Here, it is advantageous, when the elastomeric or rubber type surface portion at least slightly protrudes from the inside wall section of upper and/or lower clamping member. This way, a direct frictional contact between first and/or second housing components and the elastomeric or rubber type surface portion of upper and/or lower clamping member can be attained. Moreover, transferring the first and/or second clamping member into their locked configuration can be accompanied by a respective squeezing or clamping of the elastic material, thereby increasing a friction effect and a respective holding or clamping force.

Moreover, the safety lock member and its upper and/or lower clamping members may comprise a thermoplastic material shaped by way of injection moulding. When upper and/or lower clamping member are provided or equipped with elastomeric or rubber type material surface portions, a two- or multi-component injection moulding process for manufacturing such safety lock members can be implemented.

According to another preferred aspect, the first and/or the second housing components comprise a substantially cylindrical geometry. Here, the safety lock member is adapted to at least partially clasp around the first and second housing components in circumferential direction. It is of further benefit, when outside facing surface portions of first and second housing components substantially flush in axial direction so as to attain a rather even and uniform surface in an interface region of first and second housing components, when the housing components are mutually interconnected or interlocked.

In still another preferred aspect, the upper and lower clamping members are releasably interconnectable by means of mutually corresponding clip members arranged on opposite circumferential end sections of upper and lower clamping member. Typically, the clip members are arranged opposite a hinge intersection interconnecting upper and lower clamping member in a pivoting configuration.

Mutually corresponding clip members of upper and lower clamping members are designed such, that a release of the clip members requires to apply a releasing force above a pre-defined threshold, which should be well above a force level that can be applied by a child. This way, the mutual corresponding clip members of upper and lower clamping member cannot be released by a child. This way, the clipping members effectively prevent and impede to dismantle the protecting second housing component from the drug delivery device.

In a further preferred aspect, the drug delivery device is designed as a pen-type injector. The device further comprises a cartridge filled with a medicament and being sealed by way of a displaceable piston. Furthermore, the drug delivery device comprises a drive mechanism comprising a piston rod to be operably engaged with the piston for the purpose of expelling a pre-defined dose of the medicament from the cartridge. However, also other drug delivery devices, such like ordinary syringes stored and/or provided in a two-component housing, e.g. for transportation purpose are also generally conceivable.

According to another independent aspect the invention further provides a safety lock member for a drug delivery device as described above. The safety lock member comprises an upper clamping member and a lower clamping member pivot mounted with respect to each other. Said upper and lower clamping members are furthermore adapted to mechanically engage with first and second housing components of a drug delivery device for inhibiting or preventing unintentional release or dismantling of said housing components.

Typically, said safety lock member can be commercially distributed without and independent of the drug delivery device. If required, even existing drug delivery devices can be retrofitted with the safety lock member.

In still another aspect, there is provided a method of inhibiting unintentional release or dismantling of a first and a second housing component of a drug delivery device as described above. Implementing of such a safety lock functionality comprises the steps of attaching an upper or a lower clamping member of a safety lock member to the first and second housing component of the drug delivery device and subsequently transferring the upper and lower clamping member into an interlock configuration, in which the safety lock member, in particular its upper and lower clamping members become positively and/or frictionally engaged with the first and second housing components.

The term “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-L eu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the pertinent art that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

In the following, two preferred embodiments of the invention will be described in greater detail by making reference to the drawings in which:

FIG. 1 schematically illustrates a drug delivery device equipped with a safety lock member in locked configuration,

FIG. 2 shows the device according to FIG. 1 with the safety lock member in release configuration,

FIG. 3 illustrates the safety lock member according to FIG. 2 in an enlarged view and

FIG. 4 shows the safety lock member in release configuration in an isolated illustration,

FIG. 5 is illustrative of the safety lock member according to FIGS. 1 to 4 in locking configuration as seen in longitudinal direction and

FIG. 6 shows the safety lock member according to FIG. 5 in a perspective illustration,

FIG. 7 is indicative of another safety lock member featuring friction pads at an inside facing side wall section of upper and lower clamping members,

FIG. 8 shows the safety lock member according to FIG. 7 in an isolated view,

FIG. 9 shows the safety lock member according to FIGS. 7 and 8 in interlock configuration along its longitudinal direction, and

FIG. 10 shows the safety lock member according to FIG. 9 in a perspective illustration.

### DETAILED DESCRIPTION

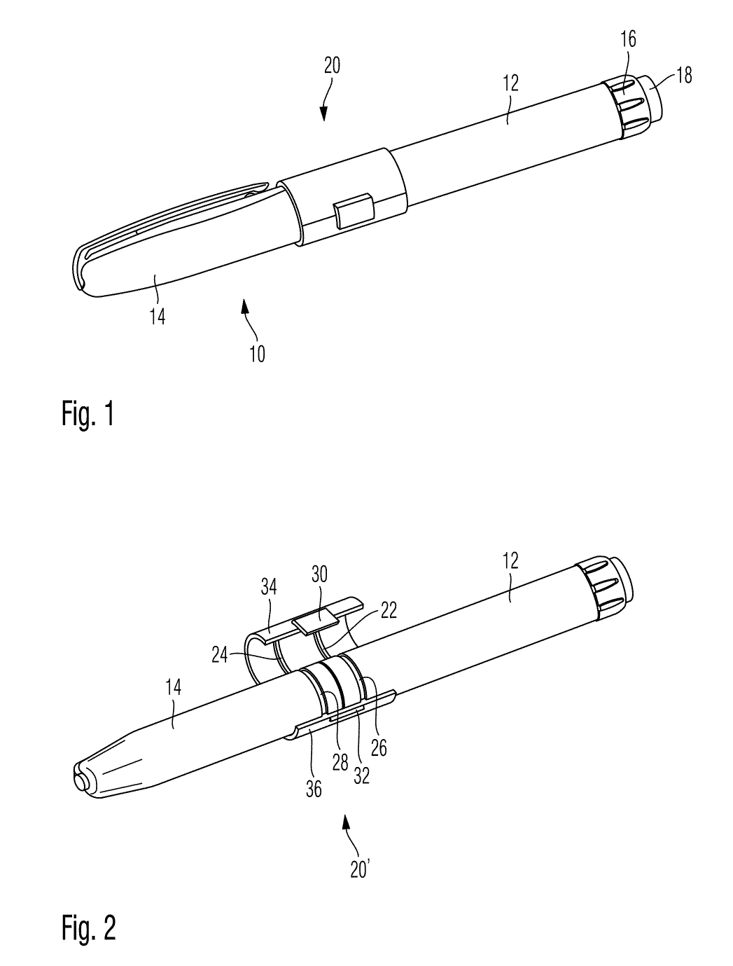

FIGS. 1 and 2 show a drug delivery device in form of a pen-type injector 10 comprising a proximal housing component 12 and a second and removable and distally located housing component 14. While the first housing component 12 serves to receive a dose dispensing or drive mechanism, the removable cap 14 serves as a protective element. When the device 10 is not in use, protective second housing component 14 should be mounted to the drug delivery device, in such a way that a cartridge holder equipped with a cartridge filled with a medicament can be effectively protected against environmental influences.

When removing the cap 14, the cartridge holder disposed underneath can be equipped with a not-illustrated piercing- or needle assembly. Then, by way of a dose dial element 16 and a dose button 18, a pre-defined dose can be set and dispensed by the drug delivery device 10.

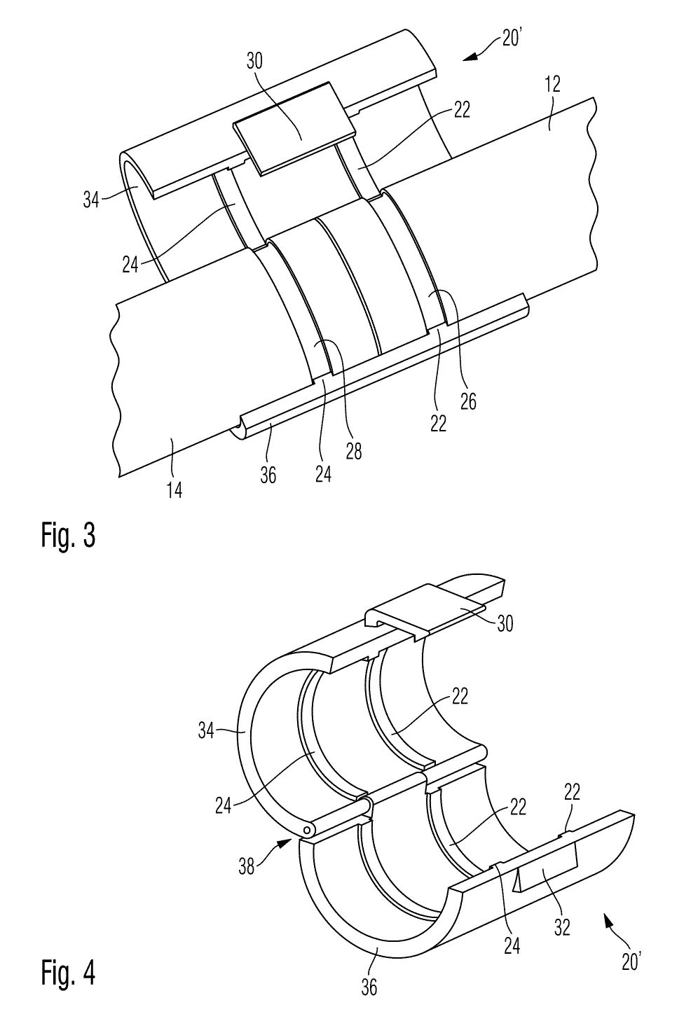

The drug delivery device 10, especially its two housing components 12, 14 are provided with a safety lock member 20 comprising an upper clamping member 34 and a lower clamping member 36. In an axial direction, that is parallel to the longitudinal direction of the drug delivery device 10, the safety lock member 20 extends across an interface section of first housing component 12 and second housing component 14 as illustrated in FIG. 3.

As further shown in FIG. 4, upper and lower clamping members 34, 36 are pivot mounted by way of a hinge 38. Diametrically opposite of the hinge 38 and its pivot axis there are provided mutually corresponding clip members 30, 32 that serve to keep the released safety lock member 20′ in a locking configuration as illustrated in FIGS. 1, 5 and 6. Here, an upper clipping member 30 extends downward from a circumferential edge portion of the upper clamping member 34. Said upper clipping member 30 overlaps with a lower clipping member 32 arranged at a corresponding axially extending edge portion of the lower clamping member 36 diametrically opposite the hinge 38.

The upper clipping member 30 comprises a latch nose 40 which is adapted to engage a lower edge portion of the lower clipping member 32 extending radially outwardly from the outward facing surface portion of the lower clamping member 36.

In particularly, the material as well as the geometry and shape of the upper clipping member 30 is selected such, that a release force above a pre-defined threshold has to be applied in order to release the mutually corresponding clip members 30, 32. The force level to be applied should be appropriately designed, such that a minimum force to release the interlock of clipping member 30, 32 is above a maximum force that can be applied by a child. This way, an effective child lock feature can be provided.

As particularly illustrated in FIGS. 3 and 4, upper and lower clamping members 34, 36 positively engage with both, first and second housing components 12, 14. For this purpose, the upper clamping member 34 comprises two circumferentially extending and radially inwardly protruding projections 22, 24 that are located at an axial distance with respect to each other. Correspondingly, also the lower clamping member 36 comprises respective radially inwardly protruding and circumferentially extending protrusions 24, 22. According to the size, the shape and position of the radially inwardly protruding projections 22, 24 of upper and lower clamping members 34, 36 also first and second housing components 12, 14 are each provided with a correspondingly shaped circumferential groove or recess 26, 28.

As illustrated in FIG. 3, recess 26 of the first housing component 12 is adapted to receive the radially inwardly projecting protrusion 22 whereas the circumferential groove 28 of the protective cap 14 matches with the projection 24 of upper and lower clamping member 34, 36.

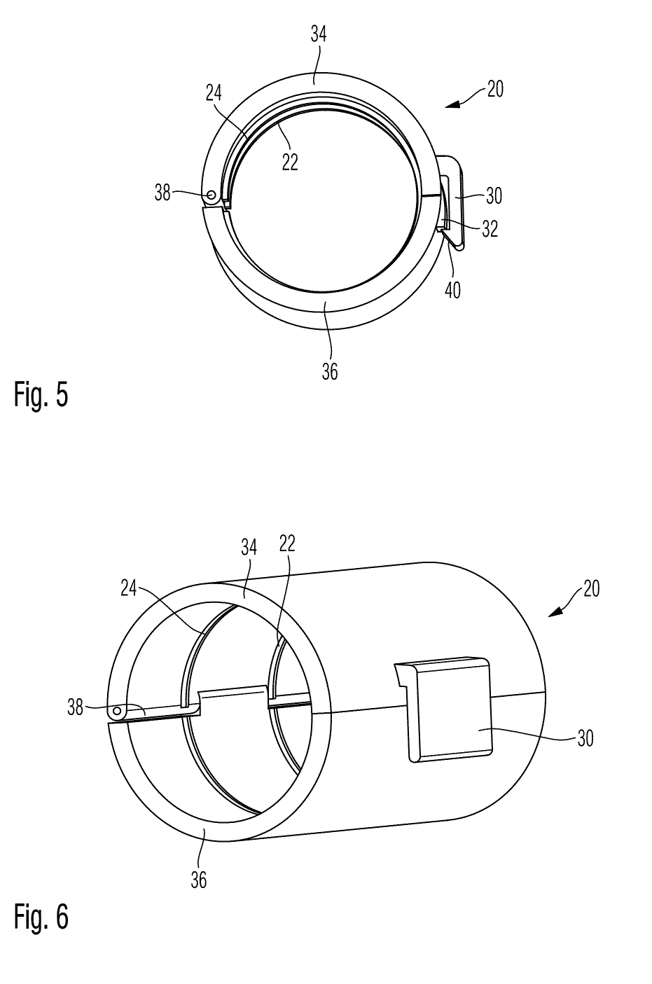

This way, a positive engagement of upper and lower clamping members 34, 36 and first and second housing components 12, 14 of the drug delivery device can be attained as soon as the safety lock member 20 gets in its locking configuration as depicted in FIGS. 5 and 6, in which mutually engaging clip members 30, 32 inhibit autonomous or unintentional dismantling or removal of first and second housing components 12, 14.

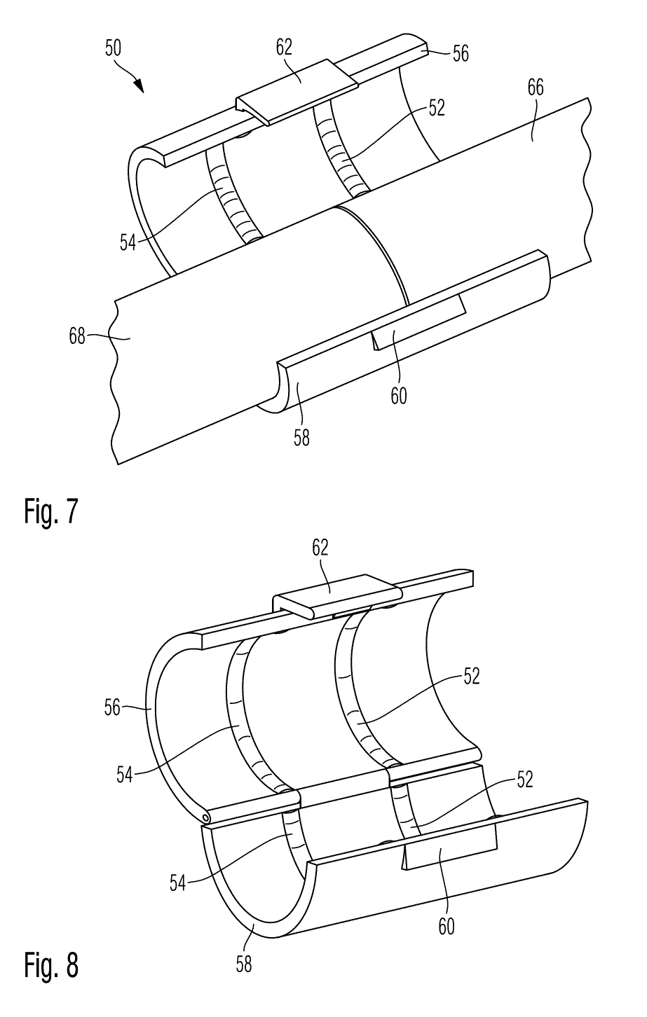

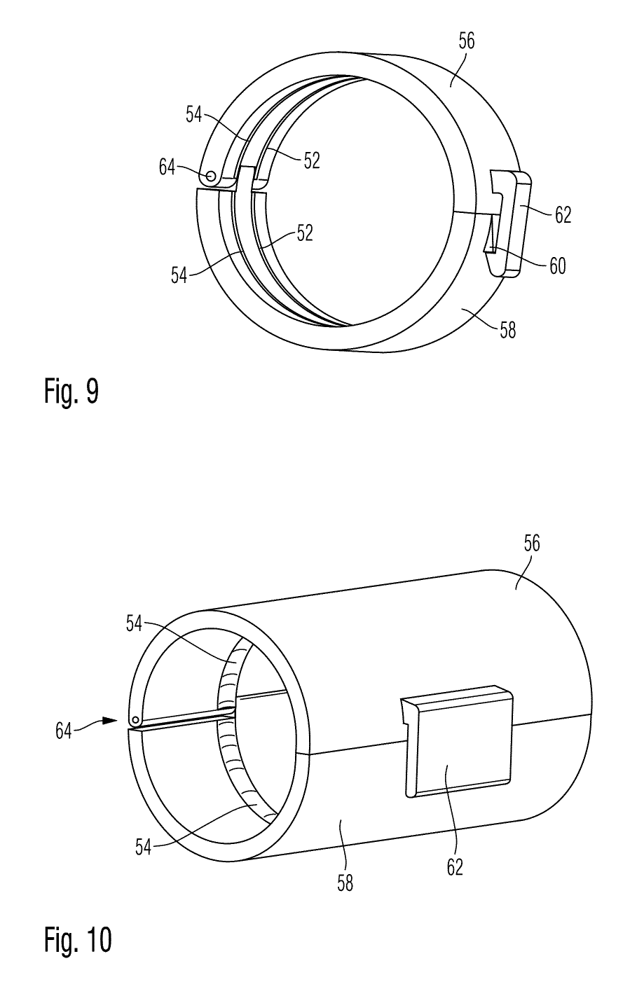

The embodiment illustrated in FIGS. 7 through 10 generally resembles the embodiment as illustrated and described with respect to FIGS. 1 to 6. But here, instead of radially protruding projections and corresponding recesses, the housing components 66, 68 of the drug delivery device are even and uniformly shaped. Hence, the housing components 66, 68 lack any circumferential protrusions or recesses. Consequently, the mutual fixing and locking mechanism provided by the safety lock member 50 and its upper and lower clamping members 56, 58 is purely based on a frictional engagement. A frictional engagement can be attained by circumferentially extending friction pads 52, 54 provided as an inside facing surface portion of the upper and lower clamping members 56, 58.

The circumferentially extending friction pads or friction strips 52, 54 may slightly protrude from an inside facing surface portion of upper and/or of lower clamping members 56, 58. The friction strips 52, 54 may comprise rubber or a comparable elastomeric material providing a sufficient gripping and/or friction effect when first and second housing components 66, 68 are enclosed by the clamping members 56, 58.

Similar to the embodiment as described with reference to FIGS. 1 to 6 also here, the upper and lower clamping members 56, 58 are pivot mounted by way of a hinge 64. Moreover, the upper clamping member 56 is equipped with a downward pointing clipping member 62 adapted to positively engage with a corresponding clipping member 60 arranged at a lateral edge of the lower clamping member 58.

The embodiment as illustrated in FIGS. 7 to 10 does not require any modifications of the housing components 66, 68 of the drug delivery device. This way, even existing drug delivery devices, such like pen-type injectors can in principle be retrofitted with a frictionally engaging safety lock member 50 as shown in FIGS. 7 through 10.

1-13. (canceled)

14. Drug delivery device for administering a dose of a medicament, comprising:
a first housing component;
a second housing component releasably interconnectable with the first housing component in a locked configuration, in which the second housing component covers a dispensing end of the device;
a safety lock member to mechanically engage with the first and with the second housing component in order to inhibit unintentional release of first and second housing component, characterized in that
the safety lock member comprises an upper and a lower clamping member adapted to receive first and second housing components, wherein upper and lower clamping members are pivot-mounted with respect to each other, with the pivot axis extending in longitudinal direction of first and/or second housing components.

15. The drug delivery device according to claim 14, wherein the safety lock member at least partially encloses the outer circumference of the first and the second housing components when in locking configuration.

16. The drug delivery device according to claim 14, wherein the upper and lower clamping member are releasably interconnectable in the locking configuration.

17. The drug delivery device according to claim 14, wherein the upper and/or the lower clamping member are positively engaged with the first housing component and with the second housing component when in interlock configuration.

18. The drug delivery device according to claim 17, wherein the upper and/or lower clamping member comprise at least two inwardly protruding protrusions and/or outwardly extending recesses adapted to engage with corresponding recesses and/or protrusions of the first and/or second housing component.

19. The drug delivery device according to claim 14, wherein the upper and/or the lower clamping member is frictionally engageable with the first housing component and with the second housing component when in interlock configuration.

20. The drug delivery device according to claim 19, wherein the upper and/or lower clamping member at an inside facing surface comprises at least one surface portion of elastomeric or rubber type material adapted to get in frictional contact with an outer surface of first and/or second housing component.

21. The drug delivery device according to claim 14, wherein the first and/or the second housing component comprise a substantially cylindrical geometry and wherein the safety lock member is adapted to at least partially clasp around the first and second housing components in circumferential direction.

22. The drug delivery device according to claim 14, wherein the safety lock member is releasably attached to the first and/or to the second housing member.

23. The drug delivery device according to claim 16, wherein the upper and lower clamping members (34, 36; 56, 58) are releasably interconnectable by means of mutually corresponding clip members (30, 32; 60, 62) arranged on opposite circumferential end sections of upper and lower clamping member (34, 36; 56, 58).

24. The drug delivery device according to claim 14, further comprising:
a cartridge filled with a medicament and being sealed by way of a displaceable piston, and
a drive mechanism comprising a piston rod to be operably engaged with the piston for expelling a pre-defined dose of the medicament from the cartridge.

25. A safety lock member for a drug delivery device according to claim 14, the safety lock member comprising:
an upper clamping member and a lower clamping member pivot mounted with respect to each other and adapted to mechanically engage with first and second housing components for inhibiting unintentional release of said housing components.

26. A method of inhibiting unintentional release of a first and a second housing component of a drug delivery device according to claim 14, comprising the steps of:
attaching an upper or a lower clamping member of a safety lock member to a first and to a second housing component and
transferring the upper and lower clamping member into an interlock configuration, in which the safety lock member is positively and/or frictionally engaged with the first and the second housing component.
